(19) United States
(12) Patent Application Publication
(10) Pub. No.: US 2003/0166583 A1
(43) Pub. Date: Sep. 4, 2003

(54) Dermal cytochrome P450 1A inhibitors and enhancers

(76) Inventors: Oliver Yoa-Pu Hu, Taipei (TW); Cheng-Huei Hsiong, Taipei (TW); Chao-Jih Wang, Taipei (TW); Li-Heng Pao, Taipei (TW)

Correspondence Address: VENABLE, BAETJER, HOWARD AND CIVILETTI, LLP, P.O. BOX 34385, WASHINGTON, DC, 20043-9998, US

(21) Appl. No.: 10/079,416

(22) Filed: Feb. 22, 2002

(51) Int. Cl. A61K 31/7048; A61K 31/56; A61K 31/57; A61K 31/35; A61K 31/353; A61K 31/11; A61K 31/23

(52) U.S. Cl. 514/27; 514/169; 514/177; 514/456; 514/552; 514/693; 514/699

(57) ABSTRACT

The present invention provides dermal cytochrome P450 1A (CYP1A) inhibitors, which include free base or pharmacologically acceptable salt of (−)-epicatechin, (+)-epicatechin, (+)-limonene, 3-phenylpropyl acetate, α-naphthoflavone, apigenin, baicalein, baicalin, β-myrcene, catechin, β-naphthoflavone, cineole, daidzein, daidzin, diosmin, ergosterol, formononetin, gallic acid, genistein, glycyrrhizin, glycyrrhizic acid, hesperetin, hesperidin, isoquercitrin, kaempferol, lauryl alcohol, luteolin, luteolin-7-glycoside, narigenin, narigin, nordihydroguaiaretic acid, oleanolic acid, paeoniflorin, quercetin, quercitrin, rutin, swertiamarin, terpineol, trans-cinnamaldehyde, trans-cinnamic acid, umbelliferone, genkwanin, homoorientin, isovitexin, neohesperidin, wongonin, capillarisin, liquiritin, ethyl myristate, poncirin, and ursolic acid. The CYP1A inhibitors can be co-administered with compounds with first-pass effect such as dermatological drugs to improve the bioavailability of the drugs. The present invention also provides dermal CYP1A enhancers, which include (+)-catechin, (−)-epicatechin, (+)-epicatechin, (+)-limonene, 3-phenylpropyl acetate, apigenin, baicalein, baicalin, β-myrcene, cineole, daidzein, daidzin, diosmin, ergosterol, formononetin, gallic acid, glycyrrhizin, hesperidin, isoquercitrin, kaempferol, lauryl alcohol, luteolin, luteolin-7-glycoside, narigin, nordihydroguaiaretic acid, paeoniflorin, protocatechuic acid, quercetin, quercitrin, rutin, swertiamarin, terpineol, trans-cinnamic acid, umbelliferone, and umbellic acid.

### FIELD OF THE INVENTION

The present invention relates to chemical compounds, which inhibit or enhance dermal cytochrome P450 1A (CYP1A) enzymatic activity. The preferred examples of the inhibitors of CYP 1A include free base or pharmacologically acceptable salt of kaempferol, luteolin-7-glycoside, terpineol, α-naphthoflavone, β-naphthoflavone, and hesperetin. The CYP1A inhibitors can be co-administered with dermatological drugs to improve the bioavailability and suppress the first-pass effect of the dermatological drugs. The preferred dermatological drug is retinoid, most favorably retinoic acid. The present invention also provides dermal CYP1A enhancers. The preferred CYP1A enhancers include (−)-epicatechin, cineole, narigin, and protocatechuic acid. The dermal CYP1A enhancers improve the CYP1A enzymatic activity so as to reduce the bioavailability of the drugs.

### BACKGROUND OF THE INVENTION

Cytochrome P450 is a heme-containing protein which was discovered by its unusually reduced carbon monoxide difference spectrum that has an absorbance at 450 nm, which is caused by a thiolate anion acting as the fifth ligand to the heme. The most common reaction catalyzed by cytochrome P450 is hydroxylation, often of a lipophilic substrate. Thus, cytochrome P450 proteins are frequently called hydroxylases.

Cytochrome P450 proteins can perform in a wide spectrum of reactions including N-oxidation, sulfoxidation, epoxidation, N-, S-, and O-dealkylation, peroxidation, deamination, desulfuration, and dehalogenation. In bacteria, these proteins are soluble and are approximately 400 amino acids in length. In eukaryotes, P450 proteins are larger and are about 500 amino acids in length. In addition, P450 proteins in eukaryotes are usually membrane bound through an N-terminal hydrophobic peptide and other less well understood contacts. The two locations of cytochrome P450 in eukaryotes are the endoplasmic reticulum membrane and the mitochondrial inner membrane, which, collectively, are referred to as “microsomes.”

Cytochrome P450 has been proven to be the major enzyme responsible for the first pass metabolism. The first-pass effect of drugs is referred to as the process of drug degradation during a drug's transition from site of entry (such as initial ingestion) to circulation in the blood stream. The first-pass effect affects bioavailability of a drug. Clinically, cytochrome P450 not only increases the first-pass metabolism in a large scale, but also magnifies the therapeutic effect as well as side effects of the drug because of drug interactions.

There are more than 1500 known P450 sequences which are grouped into families and subfamilies. CYP is the root for cytochrome P450, and CYP1 family designates one of the animal CYPs. CYP1A is present in human organs such as skin, intestine, and livers, and plays an important role in metabolism of highly variable molecules to affect the bioactivity of such molecules. The CYP1A subfamily contains two members, CYP1A and CYP1A2. CYP1A and CYP1A2 are best known for their activities to catalyze the activation of procarcinogens such as polycyclic aromatic hydrocarbons and aromatic N-arylamines, respectively, enhancing chemically induced carcinogenesis in animals and in humans. (Ioannides et al., Drug Metab. Rev. (1993), 25:453-484). CYP1A2 is constitutively present in human liver. (Sesardic et al., Carcinogenesis (1990), 11:1183-1188). However, whether CYP1A1 is also constitutively expressed in human liver is disputed, even though CYP1A1 is known to be expressed in skin. (Li et al., Carcinogenesis (1995), 16:519-524). Several studies have demonstrated that expression of CYP1A1 can be induced by xenobiotics in rat skin as well as cultured keratinocytes. (Mukhtar et al., Drug metab. Dispos. (1981), 9:311-314; Bickers et al., J. Pharmacol. Exp. Ther., (1992), 223:163-168). Regulation of CYP1A1 expression is thought to play a critical role in carcinogenesis, since many chemicals which induce skin CYP1A1 expression are also initiators of skin tumors in man. (Kinoshita et al., Cancer Res.(1972), 32:1329-1339).

The skin, as the organ in the human body with the biggest area, is the first barrier against external harmful conditions. CYP1A, which is located beneath the skin, adjusts and controls the metabolism and bioavailability of the drugs administered through contact with skin. Thus, an appropriate CYP1A activity inhibitor would effectively inhibit the enzyme activity so that the following advantages can be obtained: First, drugs can be administered cutaneously to avoid high first pass metabolism and decrease this clinical trouble. Second, the side effect and dosage of the so-called highly variable drug would be decreased. Third, the toxicity of carcinogenic metabolite caused by CYP1A activity would be decreased. Finally, new combinations of drugs and new administration route of drugs become available.

Efforts have been made to identify the inhibitors of CYP1A enzymatic activities in order to increase bioavailability of a drug, reduce drug interactions due to CYP1A, and serve as chemopreventor to avoid carcinogenesis. However, as of this time, most of the studies relating to CYP1A are concentrated on findings of CYP1A inhibitors in the liver or hepatic cell lines (see, e.g., Nielsen et al., Xenobiotica (1998) 28:389-401; Paloni et al., Cancer letters (1999), 145:35-42; Yamazaki et al., J. Chromatography (1999), 721:13-19; Maenpaa et al., Biochem. Pharmacol. (1993), 45:1035-1042). Little has been reported on inhibition of dermal CYP1A.

The invention to be presented in the following section is devoted to the findings of dermal CYP1A inhibitors and enhancers which can be used to improve or reduce the bioavailability of the dermatological drugs and as chemopreventors to prohibit the conversion of procarcinogens into potent carcinogens via CYP1A activity.

### SUMMARY OF THE INVENTION

The present invention provides a dermal cytochrome P450 1A (CYP1A) inhibitor which is a free base or pharmacologically acceptable salt of at least one of the following compounds: (−)-epicatechin, (+)-epicatechin, (+)-limonene, 3-phenylpropyl acetate, α-naphthoflavone, apigenin, baicalein, baicalin, β-myrcene, catechin, β-naphthoflavone, cineole, daidzein, daidzin, diosmin, ergosterol, formononetin, gallic acid, genistein, glycyrrhizin, glycyrrhizic acid, hesperetin, hesperidin, isoquercitrin, kaempferol, lauryl alcohol, luteolin, luteolin-7-glycoside, narigenin, narigin, nordihydroguaiaretic acid, oleanolic acid, paeoniflorin, quercetin, quercitrin, rutin, swertiamarin, terpineol, trans-cinnamaldehyde, trans-cinnamic acid, umbelliferone, genkwanin, homoorientin, isovitexin, neohesperidin, wongonin, capillarisin, liquiritin, ethyl myristate, poncirin, and ursolic acid. The dermal CYP1A inhibitors are anti-first-pass effect compounds.

The preferred dermal cytochrome P450 1A (CYP1A) inhibitors are kaempferol, luteolin-7-glycoside, terpineol, α-naphthoflavone, β-naphthoflavone, and hesperetin. kaempferol, at a concentration of about 100 μM, inhibits more than 90% of dermal microsomal CYP1A activity. Luteolin-7-glycoside, at a concentration of about 100 μM, inhibits more than 90% of dermal microsomal CYP1A activity. Terpineol, at a concentration between about 1 μM and about 100 μM, inhibits more than 75% of dermal microsomal CYP1A activity, and, at a concentration between about 10 μM, inhibits more than 90% of dermal microsomal CYP1A activity. α-naphthoflavone, at a concentration of between about 10 μM and about 100 μM, inhibits more than 90% of dermal microsomal CYP1A activity. β-naphthoflavone, at a concentration of about 100 μM, inhibits more than 90% of dermal microsomal CYP1A activity. Hesperetin, at a concentration of about 1 μM, inhibits more than 90% of dermal microsomal CYP1A activity.

The CYP1A inhibitors can be applied to skin alone or co-administered with a dermatological drug in the forms of lotion, cream, suspension or drops. The preferred dermatological drug is retinoid, and most favorably, retinoic acid. The CYP1A inhibitors can be topically applied to patient with skin cancer as chemopreventors.

The present invention also provides dermal CYP1A enhancers which include free base or pharmacologically acceptable salt of at least one of the following compounds: (+)-catechin, (−)-epicatechin, (+)-epicatechin, (+)-limonene, 3-phenylpropyl acetate, apigenin, baicalein, baicalin, O-myrcene, cineole, daidzein, daidzin, diosmin, ergosterol, formononetin, gallic acid, glycyrrhizin, hesperidin, isoquercitrin, kaempferol, lauryl alcohol, luteolin, luteolin-7-glycoside, narigin, nordihydroguaiaretic acid, paeoniflorin, protocatechuic acid, quercetin, quercitrin, rutin, swertiamarin, terpineol, trans-cinnamic acid, umbelliferone, and umbellic acid. The preferred CYP1A enhancers include (−)-epicatechin, cineole, narigin, and protocatechuic acid.

(−)-Epicatechin, at a concentration between about 10 μM and about 100 μM, enhances dermal microsomal CYP1A activity by at least 20%. Cineole, at a concentration of about 1 μM, enhances dermal microsomal CYP1A activity by about twice, and also at the concentration of about 10 μM, enhances dermal microsomal CYP1A activity by about 30%. Narigin, at a concentration of about 1 μM, enhances dermal microsomal CYP1A activity by about 50%. Protocatechuic acid, at a concentration of about 1 μM, enhances dermal microsomal CYP1A activity by about 40%, and also at the concentration of about 10 μM, enhances dermal microsomal CYP1A activity by about 20%.

### BRIEF DESCRIPTION OF THE DRAWINGS

FIG. 1 shows a time course of trans-retinoic acid (tRA) concentration (ng/ml) in plasma after transdermally applying tRA to SD rat with (●) or without (▪) co-administration of terpineol.

### DETAILED DESCRIPTION OF THE INVENTION.

The present invention provides dermal cytochrome P450 1A (CYP1A) inhibitors which suppress first-pass effect on dermatological drugs. The “first-pass effect” of drugs refers to the process of drug degradation during a drug's transition from initial ingestion or application to skin to circulation in the blood stream. The word “drug” as used herein is defined as a chemical capable of administration to an organism, which modifies or alters the organism's physiology. More preferably, the word “drug” as used herein is defined as any substance intended for use in the treatment or prevention of disease, particularly for humans. Drug includes synthetic and naturally occurring pharmaceuticals, such as those listed in Merck Index, Merck Research laboratories, Whitehouse Station, N.J.; “The Physician's Desk Reference”; “Goodman and Gilman's The Pharmacological Basis of Therapeutics”; and “The United States Pharmacopoeia, The National Formulary”. The compounds of these references are herein incorporated by reference. The word “drug” also includes compounds that have the indicated properties that are not yet discovered or available in the United States, and are pro-active, activated and metabolized forms of drugs.

One of the examples of the “first-pass effect” drug that can be combined with the CYP1A inhibitor is retinoid. Retinoid is functional and structural derivatives of retinoic acid, which has been successfully treated patients with acne, particularly nodular acne, psoriasis, disorders of Keratinion and oncology. Retinoic acid (RA) is a natural product of retinoid. It is biosynthesized and present in a multitude of human and mammalian tissues and is known to play an important rule in the regulation of gene expression, tissue differentiation and other important biological processes in mammals including humans. Retinoic acid can be metabolized into 4-hydroxyl retinoic acid due to first-pass effect. Deficiency in retinoic acid can affect the mammalian's curing capability on dermatological related diseases and cancer prevention ability.

As described in U.S. Pat. No. 6,313,107, compounds which have retinoid-like activity are well known in the art. It is generally known and accepted in the art that retinoid-like activity is useful for treating animals of the mammalian species, including humans, for curing or alleviating the symptoms and conditions of numerous diseases and conditions. In other words, it is generally accepted in the art that pharmaceutical compositions having a retinoid-like compound or compounds as the active ingredient are useful as regulators of cell proliferation and differentiation, and particularly as agents for treating skin-related diseases, including, actinic keratoses, arsenic keratoses, inflammatory and non-inflammatory acne, psoriasis, ichthyoses and other keratinization and hyperproliferative disorders of the skin, eczema, atopic dermatitis, Darriers disease, lichen planus, prevention and reversal of glucocorticoid damage (steroid atrophy), as a topical anti-microbial, as skin anti-pigmentation agents and to treat and reverse the effects of age and photo damage to the skin.

Retinoid compounds are also useful for the prevention and treatment of cancerous and precancerous conditions, including, premalignant and malignant hyperproliferative diseases such as cancers of the breast, skin, prostate, cervix, uterus, colon, bladder, esophagus, stomach, lung, larynx, oral cavity, blood and lymphatic system, metaplasias, dysplasias, neoplasias, leukoplakias and papillomas of the mucous membranes and in the treatment of Kaposi's sarcoma.

In addition, retinoid compounds can be used as agents to treat diseases of the eye, including, without limitation, proliferative vitreoretinopathy (PVR), retinal detachment, dry eye and other comeopathies, as well as in the treatment and prevention of various cardiovascular diseases, including, without limitation, diseases associated with lipid metabolism such as dyslipidemias, prevention of post-angioplasty restenosis and as an agent to increase the level of circulating tissue plasminogen activator (TPA). Other uses for retinoid compounds include the prevention and treatment of conditions and diseases associated with human papilloma virus (HPV), including warts and genital warts, various inflammatory diseases such as pulmonary fibrosis, ileitis, colitis and Krohn's disease, neurodegenerative diseases such as Alzheimer's disease, Parkinson's disease and stroke, improper pituitary function, including insufficient production of growth hormone, modulation of apoptosis, including both the induction of apoptosis and inhibition of T-Cell activated apoptosis, restoration of hair growth, including combination therapies with the present compounds and other agents such as MinoxidilR, diseases associated with the immune system, including use as immunosuppressants and immunostimulants, modulation of organ transplant rejection and facilitation of wound healing, including modulation of chelosis.

It is not uncommon for a drug that is administered to a patient orally to be given in a 5-fold or greater amount than ultimately necessary due to the degradation that occurs in the patient's body after intake. For example, in the case of the antihistamine terfenadine, 99.5% of the active ingredient ferfenadine, when given by mouth, is quickly changed to metabolites, so that the bioavailability of terfenadine is approximately 0.5%. Also, cyclosporin A, which is often administered to organ transplant patients, has a median oral bioavailability of approximately 30% and a bioavailability range of approximately 8-92% among patients (U.S. Pat. No. 6,063,809).

Although the agent(s), enzyme type(s), biological processes, etc., which are responsible for the first-pass effect have not been fully identified, research has focused on agents capable of inhibiting the cytochrmoe P450 system. Inhibition of the P450 system is a model for in vitro determination of in vivo bioavailability enhancement. See, e.g., U.S. Pat. Nos. 5,478,723, which is incorporated herein by reference. Based on the information provided by this patent, the in vivo bioavailability of a drug can be determined by in vitro analysis of cytochrome P450 enzyme activity, such as by detecting the CYP1A activity in microsomes isolated from skin.

The present invention provides inhibitors for CYP1A activity including (−)-Epicatechin, (+)-Catechin, (+)-Epicatechin, (+)-Limonene, 3-Phenylpropyl acetate, Alpha-NF, Apigenin, Baicalein, Baicalin, Beta-Myrcene, Beta-Naphthoflavone, Catechin, Cineole, Daidzein, Daidzin, Diosmin, Ergosterol, Formononetin, Gallic acid, Genistein, Glycyrrhizin, Glycyrrhizic acid, Hesperetin, Hesperidin, Isoquercitrin, Kaempferol, Lauryl alcohol, Luteolin, Luteolin-7-Glycoside, Narigenin, Narigin, Nordihydroguaiaretic acid, Oleanolic acid, Paeoniflorin, Quercetin, Quercitin, Rutin, Swertiamarin, Terpineol, Trans-Cinnamaldehyde, Trans-Cinnamic acid, Umbelliferone, Genkwanin, Homoorientin, Isovitexin, Neohesperidin, Wongonin, Capillarisin, Ursolic acid, or the pharmaceutically acceptable salts thereof, or a combination of two or more of the above, or a combination of two or more of the pharmaceutically acceptable salts thereof.

All of the CYP1A inhibitors tested not only were commercially available, but also could be extracted from herbs. For example, (1) Genkwanin, having the chemical name as narigen 7-methyl ether (2,3-dihydro-5-hydoxy-2-(4-hydroxyphenyl)-7-methoxy-4H-1-benzopyran-4-one or 4′,5-dihydroxy-7-methoxyflavanone), and having the chemical structure of:

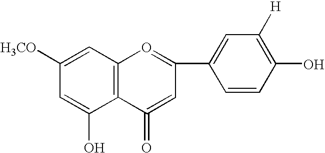

can be extracted from the flower of Daphne genkwa Sieb. et Zucc. Daphne is in the family of Thymelaeaceae. The flower of Daphne contains flavonoids such as genkwanin, hydroxygenkwanin, apigenin.

(2) Capillarisin, which is a chromone, has the chemical structure of:

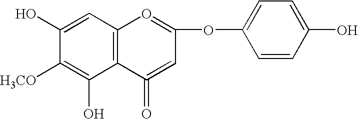

Capillarisin can be extracted from the seedling of Artemisia capillaris Thumb. Artemisia belongs to the family of Compositae. It contains essential oils such as capillin, capillene, capillone, capillarin. In addition, it contains coumarin such as esculetin, 6,7-dimethyl ether; chromone such as capillarisin and 4′-methylcapillarisin; and flavonoid such as cirsilineol and cirsimaritin.

(3) Wogonin, which is also a flavonoid, has the chemical structure of:

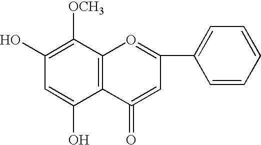

Wogonin can be extracted from the root of Scutellaria baicalensis GEORGI. Scutellaria belongs to the family of Labiatae. The root of Scutellaria baicalenesis GEORGI contains baicalin (4.3%), baicalein, wogonin (0.5%), wogonin glucuronide, oroxyline A, oroxyline A glucuronide, skullcapflavone I, skullcapflavone II, koganebananin. In addition, the root of Scutellaria baicalenesis GEORGI contains steroids such as β-sitosterol, campesterol, stigmasterol; and sugars such as sucrose, D-glucose.

(4) Baicalein and baicalin, which, like wogonin, are also extracted from the root of Scutellaria baicalensis GEORGI. The chemical structures of baicalein and baicalin are as follows:

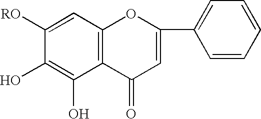

wherein R═H for baicalein and R═GlcA for baicalin.

The dermal CYP1A inhibitors can be applied alone or together with dermatological drug(s) to skin in topical formulations. The formulations suitable for topical administration include liquid or semi-liquid preparations suitable for penetration through the skin to the site of where treatment is required, such as liniments, lotions, creams, ointments or pastes, and drops suitable for administration to the eye, ear or nose.

Drops according to the present invention may comprise sterile aqueous or oily solutions or suspensions and may be prepared by dissolving the dermal CYP1A inhibitors in a suitable aqueous solution of a bactericidal and/or fungicidal agent and/or any other suitable preservative, and preferably including a surface active agent. Examples of bactericidal and fungicidal agents suitable for inclusions in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the CYP1A inhibitors. They may be made by mixing the CYP1A inhibitors in finely-divided or powdered form, alone, or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap, a mucilage, an oil of natural origin such as almond, corn, arachis, castor or olive oil, wool fat or its derivatives, or a fatty acid such as stearic or oleic acid together with an alcohol such as propylene glycol or macrogols. The formulation may incorporate any suitable surface active agent such as sorbitan esters, polyoxyethylene cellulose derivatives, or inorganic materials such as silicaceous silicas. Other ingredients such as lanolin may also be included.

It will be recognized by one of skill in the art that the optimal quantity and spacing of individual dosages of the CYP1A inhibitors of the present invention will be determined by the nature and extent of the condition being treated, the form, route, and site of administration, and the particular patients being treated, and that such optimums can be determined by conventional techniques.

The enhancers for CYP1A activity of the present invention include (+)-Catechin, (−)-Epicatechin, (+)-Epicatechin, (+)-Limonene, 3-Phenylpropyl acetate, 3-Phenylpryl acetate, Apigenin, Baicalein, Baicalin, Beta-Myrcene, Catechin, Cineole, Daidzein, Daidzin, Diosmin, Ergosterol, Formononetin, Gallic acid, Genistein, Glycyrrhizin, Glycyrrhizic acid, Hesperidin, Isoquercitrin, Kaempferol, Lauryl alcohol, Luteolin, Luteolin-7-Glucoside, Narigenin, Narigin, Nordihydroguaiaretic acid, Oleanolic acid, Paeoniflorin, Protocatechuic acid, Quercetin, Quercitrin, Rutin, Swertiamarin, Terpineol, Trans-cinnamic acid, Umbelliferone, Umbellic acid, or the pharmaceutically acceptable salts thereof, or a combination of two or more of the above compounds, or a combination of the pharmaceutically acceptable salts thereof. The above-mentioned CYP1A enhancers can induce the CYP1A activity.

The following examples are illustrative, and should not be viewed as limiting the scope of the present invention. Reasonable variations, such as those occur to reasonable artisan, can be made herein without departing from the scope of the present invention.

In particular, in Example 5 of the present invention, an in vivo study showing the effect of one of the dermal CYP1A inhibitor on the increase of bioavailability of retinoic acid (a natural product of retinoid) in rat was conducted. Similar results were obtained using other dermal CYP1A inhibitors described in the present invention. Example 5 is not to be construed as limiting to only co-administration of terpineol with retinoic acid.

### EXAMPLE 1

### Preparation of Microsomes

Nude mice were sacrificed by decapitation. The skin and liver were immediately removed and kept in ice bath or 4° C. The tissue was rinsed in 1.15% Potassium Chloride (KCl) for more than three (3) times. Connective tissue, blood, and fat were carefully removed from the tissue. Excess water content was removed from the homogenized tissue by tissue papers before the tissue was weighed.

The tissue was transferred to and homogenized in tissue homogenizer (Polytron), then, 1.15% potassium chloride solution was mixed with the homogenized tissue. The suspension of homogenized tissue in potassium chloride solution was transferred into a Teflon pestle-glass homogenizer for homogenization for six (6) times. Then, potassium chloride solution was added into the homogenized suspension to bring the volume to about four (4) times of the homogenized suspension. The diluted suspension was 5 transferred into a centrifuge tube and centrifuged at 4° C., 9000 xg, for twenty (20) minutes. The supernatant containing microsomes from skin or liver tissue and other soluble substance were removed and the volume of the supernatant was brought up to four (4) times of the weight of the tissue by adding potassium chloride solution. The diluted supernatant was transferred into a centrifuge tube and centrifuged at 4° C., 100,000 xg, for one (1) hour (Beckman L8-80M). The precipitate containing the microsomes was obtained. Potassium chloride solution was added into the precipitate to bring the volume up to four (4) times of the weight of the tissue, and the suspension was centrifuged at 4° C., 100,000 xg for one (1) hour, to obtain the washed microsomes, which were stored at −78° C. in 0.1M KH₂PO₄-K₂HPO₄ buffer (pH=7.4).

### EXAMPLE 2

### Quantitative Determination of Microsomal protein

Lowry protein assay was used to measure the protein content in the microsomes. Bovine serum albumin having concentrations of 0.1 mg/ml, 0.2 mg/ml, 0.3 mg/ml, and 0.4 mg/ml were used as standards. Alkaline copper solution of about 0.2 ml was added into each standard and skin microsome suspension diluted by water. The alkaline copper solution was prepared by mixing thoroughly 2% NaCO₃/0.1N NaOH, 1% CuSO₄·5H₂O and 2% NaK tartrate in a ratio of 100:1:1. After the alkaline copper solution was added into the standard or the microsomal suspension, the mixture was mixed thoroughly and sat at room temperature for ten (10) minutes. Then, 0.2 ml of 1N Folin-Ciocalteus phenol was added into each mixture and immediately mixed by vigorously agitating the mixture. The mixture was stayed for thirty (30) minutes. Within thirty (30) minutes, the samples were tested for absorption at 450 nm. The concentrations of bovine serum albumin standards and their O.D. were plotted for standard curve, and based on the O.D., the protein content in the microsome suspension was calculated.

### EXAMPLE 3

### Determination of Cytochrome P450 1A

Cytochrome P450 1A was quantitatively determined by carbon monoxide difference spectrum. Microsomal suspension having about 1.0-2.5 mg/ml protein was used in the experiment. Two-milliliter (2 ml) of such suspension was reduced by reacting with about 1 mg sodium dithionite. One milliliter (1 ml) of such reacted solution was transferred by titrate pipette into each reference and sample vial. UV-visible spectrum photometer was first standardized for basal lines at 350-550 nm; then, sample vial was removed from the spectrum photometer and slowly injected with carbon monoxide (CO) for fifteen (15) seconds. The sample vial was placed back into the spectrum photometer for scanning and recording of the difference between maximum absorption around 450 nm and at 490 nm.

### EXMPLE 4

### Determination of Enzymatic Activity of 7-ethoxyresorufin-O-deethylase (CYP1A) and 7-methoxyresorufin-O-demethylase (CYP1A2) and Determination of Inhibition or Enhancement of CYP1A Enzymatic Activity

The enzymatic activity of CYP1A was determined based on the activity of 7-ethoxyresorufin-O-deethylase (i.e., for CYP1A) and 7-methoxyresorufin-O-demethylase (i.e., CYP1A2). The microsomal suspension having protein of about 0.125 mg was reacted in 0.1M Hepes Buffer (pH=7.8), 5.7 mM Glucose-6-phosphate, 1.4 unit of Glucose-6-phosphate dehydrogenase, 5.7 mM Magnesium chloride, 1.8 mg/ml bovine serum albumin, 1.9 μM 7-ethoxyresorufin or 7-methoxyresorufin, and 12.9 μl CYP1A1 or CYP1A2 activity inhibitor or enhancer, and the total volume of the reaction mixture was 1.2879 ml. Except for the blank group, the reaction was triggered by adding 0.54 μM NADPH. In the test tube (16×100 mm), the reaction was carried out for ten (10) minutes at constant temperature (37° C.) with vibration and without light Methanol 2.5 ml was added into the reaction mixture to stop the reaction. Protein precipitate was removed from the reaction mixture by centrifuging the reaction mixture at 180 xg for ten (10) minutes. The supernatant was tested for florescent intensity with 550 nm as the excitation wavelength and 585 nm the emission wavelength. The florescent intensity was compared with 0.01 μM-0.1 μM standard Rhodamine B solution to obtain the reading of the CYP1A enzyme activity.

Results:

The inhibitory and enhancing effects of various compounds on dermal CYP1A activity is summarized in Table 1:

TABLE 1
Inhibitory and Enhancing Effects of Various Compounds on Dermal
CYP1A Activity

| Compound Concentration | | Compound Concentration | | Compound Concentration | |
|---|---|---|---|---|---|
| 1 μM | | 10 μM | | 100 μM | |
| | activity* | | activity* | | activity* |
| name | (%) | name | (%) | name | (%) |
| Hesperetin | 6.89 | Terpineol | 2.99 | Kaempferol | 0 |
| Terpineol | 23.55 | Alpha-NF | 8.03 | Alpha-NF | 0.83 |
| Oleanolic acid | 23.98 | Luteolin | 19.41 | Luteolin-7- | 2.53 |
| | | | | glucoside | |
| Beta-NF | 27.41 | Kaempferol | 22.35 | Beta-NF | 3.76 |
| 3-Phenyl | 28.29 | Beta-NF | 24.00 | Quercetin | 8.37 |
| propyl acetate | | | | | |
| Narigenin | 29.41 | Hesperetin | 24.63 | Luteolin | 10.45 |
| Kaempferol | 33.73 | Oleanolic acid | 25.74 | Terpineol | 13.18 |
| Alpha-NF | 34.65 | Trans- | 28.88 | Narigenin | 17.26 |
| | | cinnamaldehyde | | | |
| (+)-Limonene | 50.30 | Narigenin | 34.90 | Trans- | 19.83 |
| | | | | cinnamaldehyde | |
| Luteolin | 59.11 | Quercetin | 41.85 | Diosmin | 32.94 |
| Hesperidin | 62.61 | Baicalein | 48.84 | Genistein | 35.40 |
| Diosmin | 64.34 | Hesperidin | 52.72 | Isoquercitrin | 44.18 |
| Baicalein | 64.73 | Umbelliferone | 52.73 | Oleanolic acid | 46.71 |
| (+)- | 65.55 | Diosmin | 54.26 | Ergosterol | 52.24 |
| Epicatechin | | | | | |
| Formononetin | 67.03 | Ergosterol | 57.32 | Lauryl alcohol | 52.43 |
| Quercetin | 67.90 | Genistein | 60.22 | Baicalein | 55.82 |
| Umbelliferone | 72.54 | Isoquercitrin | 66.97 | Hesperidin | 57.75 |
| Genistein | 77.37 | Daidzein | 71.73 | Rutin | 59.21 |
| Daidzein | 77.53 | Quercitrin | 77.08 | Nordihydro- | 59.45 |
| | | | | guaiaretic acid | |
| (+)-Catechin | 78.10 | Beta-Myrcene | 78.17 | Hesperetin | 66.01 |
| Trans-cinna | 82.76 | Baicalin | 82.85 | Daidzin | 70.44 |
| maldehyde | | | | | |
| (−)-Epicatechin | 85.69 | Daidzin | 84.23 | Beta-Myrcene | 73.64 |
| Quercitrin | 87.84 | (+)-Epicatechin | 86.15 | Quercitrin | 75.00 |
| Lauryl alcohol | 88.20 | Luteolin-7- | 88.05 | Umbelliferone | 75.75 |
| | | glucoside | | | |
| Baicalin | 90.14 | 3-Phenyl propyl | 89.40 | Baicalin | 75.91 |
| | | acetate | | | |
| Paeoniflorin | 90.95 | (+)-Limonene | 89.81 | Apigenin | 76.38 |
| Rutin | 94.11 | (+)-Catechin | 91.97 | Daidzein | 77.18 |
| Swertiamarin | 98.52 | Swertiamarin | 96.55 | Narigin | 77.31 |
| Glycyrrihizin | 98.89 | Nordihydro | 96.64 | 3-Phenyl propyl | 85.04 |
| | | guaiaretic acid | | acetate | |
| Beta-Myrcene | 101.19 | Apigenin | 97.22 | Formononetin | 85.87 |
| Gallic acid | 102.10 | Lauryl alcohol | 97.22 | Trans-cinnamic | 86.62 |
| | | | | acid | |
| Apigenin | 103.47 | Rutin | 98.43 | Gallic acid | 88.23 |
| Luteolin-7- | 105.07 | Glycyrrihizin | 102.20 | Cineole | 96.40 |
| glucoside | | | | | |
| Nordihydro | 110.23 | Paeoniflorin | 104.74 | (+)-Catechin | 99.99 |
| guaiaretic acid | | | | | |
| Daidzin | 110.92 | Narigin | 103.30 | Protocatechuic | 101.94 |
| | | | | acid | |
| Isoquercitrin | 112.30 | Gallic acid | 106.85 | Paeoniflorin | 103.88 |
| Trans- | 117.33 | Formononetin | 109.42 | Glycyrrihizin | 106.70 |
| cinnamic acid | | | | | |
| Protocatechuic | 138.35 | Trans-cinnamic | 110.24 | (+)-Epicatechin | 105.09 |
| acid | | acid | | | |
| Narigin | 149.99 | Protocatechuic | 121.12 | Swertiamarin | 105.24 |
| | | acid | | | |
| Cineole | 199.40 | (−)-Epicatechin | 121.77 | (+)-Limonene | 107.26 |
| | | Cineole | 129.93 | (−)-Epicatechin | 129.82 |

*activity is shown as percentage as compared to the activity of the control group.

Each data point shown in Table 1 is the mean value of at least 3 repeats. As shown in Table 1, the compounds that had shown inhibition effects on dermal CYP1A activity include (−)-epicatechin, (+)-catechin, (+)-epicatechin, (+)-limonene, 3-phenylpropyl acetate, α-naphthoflavone, apigenin, baicalein, baicalin, β-Myrcene, β-naphthoflavone, cineole, daidzein, daidzin, diosmin, ergosterol, formononetin, gallic acid, genistein, glycyrrhizin, hesperidin, hesperetin, isoquercitrin, kaempferol, lauryl alcohol, luteolin, luteolin-7-glucoside, narigenin, narigin, nordihydroguaiaretic acid, oleanolic acid, paeoniflorin, quercetin, quercitrin, rutin, swertiamarin, terpineol, trans-cinnamaldehyde, trans-cinnamic acid, and umbelliferone. Among these inhibitors, kaempferol, luteolin-7-glycoside, terpineol, β-naphthoflavone, and hesperetin demonstrated the strongest inhibitory effect on CYP1A, particular at certain concentration.

Some other compounds, which included genkwanin, homoorientin, isovitexin, neohesperidin, wongonin, capillarisin, liquiritin, ethyl myristate, poncirin, and ursolic acid, and which are not listed in Table 1, also demonstrated strong inhibitory effects on dermal CYP1A activity.

The results as shown in Table 1 also demonstrate that the inhibitory effects of the tested compounds varied due to the concentrations of the compounds used in the study. In other words, a compound could demonstrate inhibitory effect at one concentration but could exhibit no inhibitory effect or enhancing effect at a different concentration. In addition, for some of the compounds tested, the inhibitory or enhancing effect was not dose-dependent, meaning that there was no positive correlation between the dosage used and the CYP1A inhibitory or enhancing activity.

For example, in the case of α-naphthoflavone, when α-naphthoflavone was at a concentration of 1 μM, the dermal CYP1A activity was about 35% of the control. When the concentration of α-naphthoflavone increased to 10 μM, the dermal CYP1A activity was reduced to about 8%. When the concentration of α-naphthoflavone was increased to 100 μM, the dermal CYP1A activity was further reduced to about 0.8% of the control. This indicates that the inhibitory effect of α-naphthoflavone was dose-dependent and the more the α-naphthoflavone was used, the better inhibitory effect was received.

Kampferol was another example of CYP1A inhibitor which demonstrated dose-dependent effect on CYP1A inhibition. When kampferol was at a concentration of 1 μM, the dermal CYP1A activity was about 34%. When the concentration of kampferol increased to 10 μM, the dermal CYP1A activity was reduced to about 22%. When the concentration of α-naphthoflavone was increased to 100 μM, the dermal CYP1A activity was further reduced to about 0% of the control.

However, in the case of terpineol, when terpineol was at a concentration of 1 μM, the dermal CYP1A activity was about 24%. When the concentration of terpineol increased to 10 μM, the dermal CYP1A activity was reduced to about 3%. But when the concentration of terpineol was increased to 100 μM, there was no further reduction or plateau of the dermal CYP1A activity, instead, the CYP1A activity was back to about 13%. This indicates that the inhibitory effect of terpineol was not dose-dependent.

Also, in the case of hesperetin, when hesperetin was at a concentration of 1 μM, the dermal CYP1A activity was about 7% of the control. When the concentration of terpineol increased to 10 μM, the dermal CYP1A activity increased to about 25%. When the concentration of terpineol increased to 100 μM, the dermal CYP1 A activity increased to about 66%. This indicates that the higher the hesperetin concentration, the lower the inhibitory effect of hesperetin.

In addition, some compounds, when used at different concentrations, might have different effects. Some compounds achieved the most effective inhibition when the concentration was at 1 μM: such as hesperetin (which inhibited about 93% of CYP1A activity), oleanolic acid (which inhibited about 76% of CYP1A activity), 3-phenylpropyl acetate (which inhibited about 72% of CYP1A activity), (+)-limonene (which inhibited about 51% of CYP1A activity), (+)-epicatechin (which inhibited about 34% of CYP1A activity), formononetin (which inhibited about 33% of CYP1A activity), and (+)-catechin (which inhibited about 22% of CYP1A activity).

Some compounds achieved the most effective inhibition at the concentration of 10 μM. Such compounds included terpineol (which inhibited about 97% of CYP1A activity), baicalein (which inhibited about 47% of CYP1A activity), hesperidin (which inhibited about 47% of CYP1A activity), umbelliferone (which inhibited about 47% of CYP1A activity), and daidzein (which inhibited about 28% of CYP1A activity).

Finally, some compounds achieved the most effective inhibition at concentration of 100 μM. Such compounds include kaempferol (which inhibited about 100% of CYP1A activity), α-naphthoflavone (which inhibited about 99% of CYP1A activity). luteolin-7-glucoside (which inhibited about 97% of CYP1A activity), β-naphthoflavone (which inhibited about 96% of CYP1A activity), quercetin (which inhibited about 92% of CYP1A activity), luteolin (which inhibited about 90% of CYP1A activity), narigenin (which inhibited about 83% of CYP1A activity), trans-cinnamaldehyde (which inhibited about 80% of CYP1A activity), diosmin (which inhibited about 67% of CYP1A activity), genistein (which inhibited about 65% of CYP1A activity), isoquercitrin (which inhibited about 56% of CYP1A activity), lauryl alcohol (which inhibited about 48% of CYP1A activity), rutin (which inhibited about 41% of CYP1A activity), nordihydroguaiaretic acid (which inhibited about 41% of CYP1A activity), daidzin (which inhibited about 30% of CYP1A activity), β-myrcene (which inhibited about 26% of CYP1A activity), quercitrin (which inhibited about 25% of CYP1A activity), baicalin (which inhibited about 24% of CYP1A activity), apigenin (which inhibited about 24% of CYP1A activity), narigin (which inhibited about 23% of CYP1A activity), trans-cinnamic acid (which inhibited about 13% of CYP1A activity), and gallic acid (which inhibited about 12% of CYP1A activity).

Table 1 also shows that some compounds, at certain concentrations, had demonstrated enhancing effects on dermal CYP1A activity. These compounds included (−)-epicatechin, (+)-catechin, (+)-epicatechin, (+)-limonene, apigenin, β-Myrcene, cineole, daidzin, formononetin, gallic acid, glycyrrhizin, isoquercitrin, lauryl alcohol, luteolin-7-glucoside, narigin, nordihydroguaiaretic acid, paeoniflorin, protocatechuic acid, rutin, swertiamarin, trans-cinnamic acid. Among these compounds, cineole, narigin, protocatechuic acid, and (−)-epicatechin had demonstrated the strongest enhancing effect on CYP1A activity.

Again, the enhancing effects of the above compounds did not necessarily and positively correlated with the dosage used in the test. For example, in the case of cineole, 1 μM of cineole demonstrated almost twice enhancement of the CYP1A activity. However, when cineole was at 10 μM, the enhancing effect reduced to about 30%. When cineole was at a concentration of 100 μM, cineole showed an inhibitory effect on CYP1A activity (about 5% inhibitory effect on CYP1A activity). Similar results as that of cineole was also found in narigin and protocatechuic acid. As for (−)-epicatechin, when 1 μM of (−)-epicatechin was added to dermal microsomal CYP1A, (−)-epicatechin displayed an inhibitory activity (inhibited about 24% of the CYP1A activity). However, when 10 μM of (−)-epicatechin was added to dermal microsomal CYP1A, (−)-epicatechin became an enhancing agent for CYP1A (enhanced 22% of the CYP1A activity). 100 μM of(−)-epicatechin further enhanced CYP1A activity to about 30%.

A study using nude mice liver microsomal CYP1A as model to detect various compounds on inhibitory and/or enhancing effects of liver CYP1A activity was also conducted. The purpose of this study was (1) to compare the inhibitory and/or enhancing effects of the same compounds on the dermal CYP1A with those of the liver CYP1A, and (2) to confirm that the inhibitory and/or enhancing effects of the same compounds on the dermal CYP1A are drastically different from those of the liver CYP1A.

The results are shown in Table 2.

TABLE 2
Inhibitory and Enhancing Effects of Various Compounds on Liver CYP1A
Activity

| Compound Concentration | | Compound Concentration | | Compound Concentration | |
|---|---|---|---|---|---|
| 1 μM | | 10 μM | | 100 μM | |
| | activity* | | activity* | | activity* |
| name | (%) | name | (%) | name | (%) |
| Alpha-NF | 32.91 | Alpha-NF | 11.06 | Trans-cinna | 4.61 |
| | | | | maldehyde | |
| Beta-NF | 75.00 | Trans-cinna | 20.93 | Alpha-NF | 7.77 |
| | | maldehyde | | | |
| Trans-cinna | 76.00 | Beta-NF | 22.78 | Beta-NF | 13.79 |
| maldehyde | | | | | |
| Beta-Myrcene | 82.00 | Kaempferol | 68.93 | Kaempferol | 18.05 |
| (+)-Catechin | 90.00 | Quercetin | 86.70 | Quercetin | 19.17 |
| Oleanolic acid | 94.00 | Hespendin | 87.38 | Luteolin | 33.39 |
| Terpineol | 96.00 | Baicalin | 90.79 | Narigenin | 41.64 |
| Isoquercitrin | 99.00 | (+)-Limonene | 93.10 | Diosmin | 43.78 |
| Hesperidin | 99.65 | Terpineol | 97.07 | Apigenin | 53.49 |
| Kaempferol | 100.11 | Diosmin | 97.26 | Oleanoic acid | 87.26 |
| (+)-Limonene | 100.70 | Beta-Myrcene | 98.05 | Glycyrrhizin | 89.10 |
| Ergosterol | 103.36 | Oleanolic acid | 99.89 | Quercitrin | 89.15 |
| Baicalin | 106.19 | Glycyrrhizin | 99.92 | Rutin | 90.68 |
| Diosmin | 108.33 | Cineole | 99.93 | Cineole | 94.70 |
| Quercitrin | 108.58 | Trans- | 103.17 | Isoquercitrin | 95.74 |
| | | cinnamic acid | | (50) | |
| Genistein | 109.12 | Isoquercitrin | 103.60 | Ergosterol | 97.68 |
| Trans- | 109.22 | Luteolin | 103.61 | Beta-Myrcene | 100.37 |
| cinnamic acid | | | | | |
| Daidzein | 109.37 | Quercitrin | 103.61 | Lauryl alcohol | 100.62 |
| Luteolin-7- | 109.79 | Ergosterol | 104.33 | Trans- | 101.33 |
| Glucoside | | | | cinnamic acid | |
| Daidzin | 110.31 | Narigenin | 107.17 | (+)-Limonene | 101.61 |
| Baicalein | 110.67 | Genistein | 107.88 | Terpineol | 102.15 |
| Lauryl alcohol | 112.69 | Daidzin | 111.54 | Hesperidin | 102.94 |
| Quercetin | 113.87 | Daidzein | 111.86 | Gallic acid | 105.13 |
| Luteolin | 114.52 | Baicalein | 114.18 | Baicalin (25) | 106.06 |
| Narigenin | 116.45 | Lauryl alcohol | 115.32 | Protocatechuic | 106.88 |
| | | | | acid | |
| Cineole | 116.59 | Umbelliferone | 118.91 | Genistein | 109.12 |
| Rutin | 117.28 | Luteolin-7- | 119.53 | Daidzin | 110.67 |
| | | glucoside | | | |
| Glycyrrhizin | 118.39 | Narigin | 119.79 | (+)-Catechin | 116.48 |
| Apigenin | 118.46 | (+)-Catechin | 120.23 | Nordihydro | 118.51 |
| | | | | guaiaretic acid | |
| Protocatechuic | 119.01 | Gallic acid | 123.07 | Narigin | 120.55 |
| acid | | | | | |
| Umbelliferone | 120.45 | Protocatechuic | 123.35 | Paeoniflorin | 120.72 |
| Formononetin | 121.41 | Paeoniflorin | 124.09 | Umbellic acid | 120.92 |
| Paeoniflorin | 122.85 | Rutin | 124.11 | Luteolin-7- | 123.78 |
| | | | | glucoside | |
| (+)- | 124.29 | (+)- | 129.86 | (+)- | 130.95 |
| Epicatechin | | Epicatechin | | Epicatechin | |
| (−)-Epicatechin | 133.54 | (−)- | 139.91 | Daidzein (50) | 134.85 |
| | | Epicatechin | | | |
| Narigin | 138.88 | 3-Phenyl | 143.19 | Formononetin | 144.70 |
| | | propyl acetate | | | |
| Nordihydro | 140.37 | Formononetin | 143.91 | (−)- | 147.44 |
| guaiaretic acid | | | | Epicatechin | |
| 3-Phenyl | 151.26 | Nordihydro | 147.20 | 3-Phenyl | 156.98 |
| propyl acetate | | guaiaretic acid | | propyl acetate | |

*activity was shown by percentage as compared to the activity of the control group.

As shown in Table 2, the compounds that have demonstrated inhibitory effects on liver CYP1A activity include: (+)-limonene, α-naphthoflavone, apigenin, baicalin, β-myrcene, β-naphthoflavone, catechin, cineole, diosmin, ergosterol, glycyrrhizic acid, hesperidin, isoquercitrin, kaempferol, luteolin, narigenin, oleanoic acid, quercetin, quercitrin, rutin, terpineol, trans-cinnamaldehyde. Among the inhibitors, trans-cinnamaldehyde, β-naphthoflavone, kaempferol, quercetin, and luteolin at certain concentration demonstrated the most inhibitory effect on liver CYP1A activity.

Other than kaempferol and β-naphthoflavone, which demonstrated inhibitory effects on both dermal and liver CYP1A activities, the most effective inhibitors in dermal CYP1A (which included luteolin-7-glycoside, terpineol, α-naphthoflavone, and hesperetin) were different from those in liver CYP1A (trans-cinnamaldehyde, kaempferol, quercetin, and luteolin). Also, although kaempferol and β-naphthoflavone had inhibitory effects on both dermal CYP1A and liver CYP1A, the effectiveness of these two compounds in inhibiting dermal CYP1A and liver CYP1A was different (at 100 μM, kampferol inhibited about 100% dermal CYP1A activity and about 82% liver CYP1A activity; at 100 μM, β-naphthoflavone inhibited about 96% dermal CYP1A activity and about 86% liver CYP1A activity).

Table 2 also demonstrates that the following compounds had enhancing effect on liver CYP1A activity: (−)-epicatechin, (+)-epicatechin, (+)-limonene, 3-phenylpropyl acetate, 3-phenylpryl acetate, apigenin, baicalein, baicalin, β-myrcene, catechin, cineole, daidzein, daidzin, diosmin, ergosterol, formononetin, gallic acid, genistein, glycyrrhizic acid, hesperidin, isoquercitrin, kaempferol, lauryl alcohol, luteolin, luteolin-7-glucoside, narigin, narigenin, nordihydroguaiaretic acid, oleanolic acid, paeoniflorin, protocatechuic acid, quercetin, quercitrin, rutin, terpineol, trans-cinnamic acid, umbelliferone, and umbellic acid.

Among the enhancers, 3-phenylpropyl acetate, (−)-epicatechin, nordihydroguaiaretic acid, formononetin, and narigin at certain concentration demonstrated the most enhancing effect on liver CYP1A activity. Again, other than (−)-epicatechin and narigin, the enhancers for liver CYP1A were different from those of the dermal CYP1A enhancers, which include cineole and protocatechuic acid. However, the inhibitory/enhancing effects of narigin and (−)-epicatechin in dermal CYP1A and liver CYP1A were different. For example, 100 μM of narigin inhibited about 23% of dermal CYP1A activity but exhibited about 21% enhancing effect on liver CYP1A. Also, 1 μM of (−)-epicatechin inhibited about 14% of dermal CYP1A activity but exhibited about 34% enhancing effect on liver CYP1A activity.

Thus, the comparative study of the compound effects on dermal CYP1A and live CYP1A activities clearly demonstrated that the inhibitory/enhancing effects of the compounds on dermal CYP1A were not predicated by those on liver CYP1A.

### EXAMPLE 5

### Effect of Terpineol on All-trans Retinoic Acid After Transdermal Application to SD-Rats

Airol vanishing cream and Airol lotion from Pierre Fabre Dermo Cosmetique, France were used in this study. The active ingredient of Airol is vitamin A acid (tretinoin, which is all-trans-retinoic acid) (tRA). The Airol vanishing cream contains 0.05% by weight of tRA in a washable ointment base. The Airol lotion contains 0.05% by weight of tRA in ethyl alcohol and propylene glycol.

One day prior to the experiment, the hair at the abdominal area (about 4×6 cm²) of the SD-rats was shaved off. The SD-rats were then fasted overnight (about 8 hours). Prior to the experiment, a tubing was inserted into the neck vein of the SD-rats for intermittent collection of blood. 3 g of either the control dosage or the experimental dosage of tRA were then applied to the abnormal skin area where the hair had been shaved off previously. The control dosage of tRA contained Airol vanishing cream or lotion (0.05% of tRA) plus 0.45% of tRA powder (dissolved in 33 μl of 99% alcohol). The experimental dosage of tRA contained Airol vanishing cream or lotion (0.05% of tRA) plus 0.45% of tRA powder (dissolved in 33 μl of 99% alcohol) plus 10% by weight of terpineol.

At 0, 5, 10, 15, and 20 hours intervals, an aliquot of blood was collected in a tube containing anticoagulant (such as 1% EDTA or heparin sulfate) followed by a low speed centrifugation to obtain the plasma. To quantify the amount of tRA in plasma, about 250 μl of plasma were mixed with about 50 μl of NEM (50 mM N-ethylmaleimide) and about 50 μl of Carbozle (200 μl/ml). Then, about 500 μl of ethyl ether were added to the plasma mixture. The ethyl ether plasma mixture was gently rotated for about 30 minutes and then stored at −80° C. for about 30 minutes, followed by evaporating the ethyl ether by air.

The tRA content in the plasma sample was analyzed by High Performance Liquid Chromatography (HPLC) as follows:

The HPLC analysis used an HAIsil C-18 column (5μm, Dimenion: 250×4.6 mm). Column temperature was maintained at 50° C. The mobile phase of the column was a gradient containing (A) methanol: 0.02M ammonium=1:1, v/v (pH 6.65); (B) methanol: 0.1M ammonium=9:1 (pH 6.5). The elution gradient was described in Table 3. The flow rate was 1.0 ml/min. The total retention time was about 30 minutes.

TABLE 3
Mobile Phase Gradient for HPLC

| Time (min) | (A) (%) | (B) (%) |
|---|---|---|
| 0 | 50 | 50 |
| 10 | 10 | 90 |
| 20 | 10 | 90 |
| 21 | 50 | 50 |
| 26 | 50 | 50 |

Post run: 15 min.

The plasma sample (after ethyl ether extraction) was added to 67 μl of mobile phase (B) and 33 μl of acetonitril. The sample mixture was centrifuged at 13,000 xg for about 5 minutes. About 50 μl of the supernatant was injected to the HPLC. After the HPLC, the amount of tRN (ng/ml) in the corresponding fraction of the HPLC was quantified.

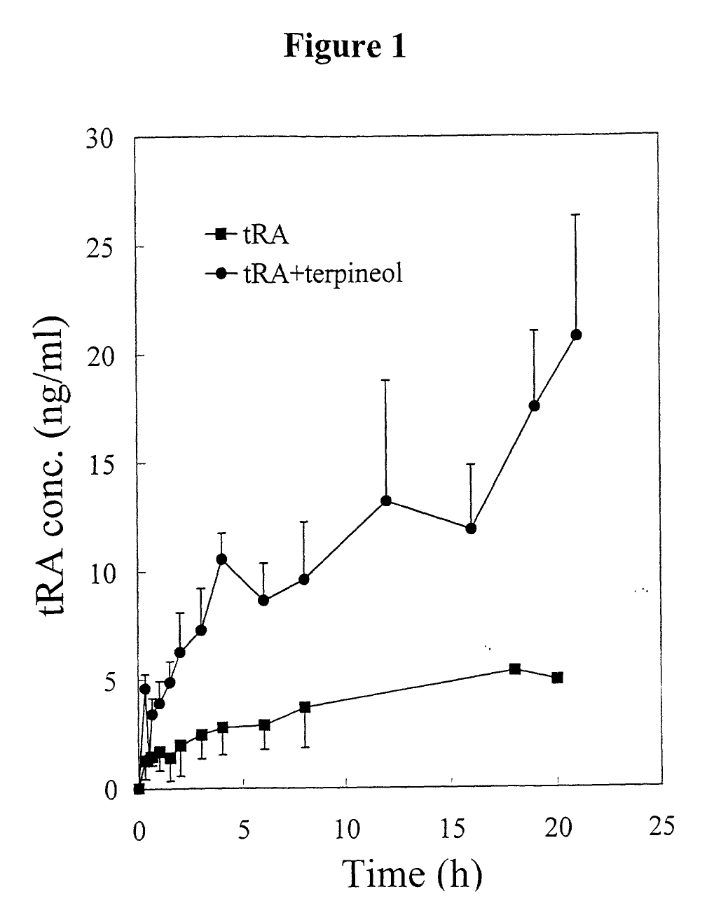

The results of this in vivo study of the effect of terpineol on serum level of tRA were shown in FIG. 1. Co-administration of terpineol with tRA through transdermal application significantly increased the tRA concentration in plasma upon time (between 0 and 20 hours), which in turn demonstrated an improved bioavailability of tRA in the circulation.

While the invention has been described by way of examples and in terms of the preferred embodiments, it is to be understood that the invention is not limited to the disclosed embodiments. On the contrary, it is intended to cover various modifications as would be apparent to those skilled in the art. Therefore, the scope of the appended claims should be accorded the broadest interpretation so as to encompass all such modifications.

We claim:

1. A dermal cytochrome P450 1A (CYP1A) inhibitor which is a free base or pharmacologically acceptable salt of at least one compound selected from the group consisting of (−)-epicatechin, (+)-epicatechin, (+)-limonene, 3-phenylpropyl acetate, α-naphthoflavone, apigenin, baicalein, baicalin, β-myrcene, catechin, β-naphthoflavone, cineole, daidzein, daidzin, diosmin, ergosterol, formononetin, gallic acid, genistein, glycyrrhizin, glycyrrhizic acid, hesperetin, hesperidin, isoquercitrin, kaempferol, lauryl alcohol, luteolin, luteolin-7-glycoside, narigenin, narigin, nordihydroguaiaretic acid, oleanolic acid, paeoniflorin, quercetin, quercitrin, rutin, swertiamarin, terpineol, trans-cinnamaldehyde, trans-cinnamic acid, umbelliferone, genkwanin, homoorientin, isovitexin, neohesperidin, wongonin, capillarisin, liquiritin, ethyl myristate, poncirin, and ursolic acid.

2. The dermal cytochrome P450 1A (CYP1A) inhibitor according to claim 1, wherein said inhibitor is at least one selected from the group consisting of kaempferol, luteolin-7-glycoside, terpineol, α-naphthoflavone, β-naphthoflavone, and hesperetin.

3. The dermal cytochrome P450 1A (CYP1A) inhibitor according to claim 1, wherein said dermal CYP1A inhibitor is an anti-first-pass-effect compound.

4. The dermal cytochrome P450 1A (CYP1A) inhibitor according to claim 1, wherein said dermal CYP1A inhibitor is co-administered with a compound having first-pass effect.

5. The dermal cytochrome P450 1A (CYP1A) inhibitor according to claim 1, wherein said compound with first-pass effect is a dermatological drug.

6. The dermal cytochrome P450 1A (CYP1A) inhibitor according to claim 5, wherein said dermatological drug is retinoid.

7. The dermal cytochrome P450 1A (CYP1A) inhibitor according to claim 6, wherein said dermatological drug is retinoic acid.

8. The dermal cytochrome P450 1A (CYP1A) inhibitor according to claim 1, wherein said CYP1A inhibitor is topically applied to patient with skin cancer.

9. The dermal cytochrome P450 1A (CYP1A) inhibitor according to claim 6, wherein said CYP1A inhibitor is topically applied to patient with skin cancer.

10. A method for treating patients with dermatological diseases comprising topically treating said patients with said dermal CYP1A inhibitor according to claim 1.

11. The method according to claim 10, wherein said dermal CYP1A is co-administered with a dermatological drug.

12. The method according to claim 11, wherein said dermatological drug is retinoid.

13. A method for treating patient with skin cancer comprising topically applying the dermal CYP1A inhibitor according to claim 1 to said patient with skin cancer.

14. The method for treating patient with skin cancer according to claim 13, wherein said dermal CYP1A inhibitor is co-administered with retinoid.

15. A dermal cytochrome P450 1A enhancer which is a free base or pharmacologically acceptable salt of at least one compound selected from the group consisting of (+)-catechin, (−)-epicatechin, (+)-epicatechin, (+)-limonene, 3-phenylpropyl acetate, apigenin, baicalein, baicalin, β-myrcene, cineole, daidzein, daidzin, diosmin, ergosterol, formononetin, gallic acid, glycyrrhizin, hesperidin, isoquercitrin, kaempferol, lauryl alcohol, luteolin, luteolin-7-glycoside, narigin, nordihydroguaiaretic acid, paeoniflorin, protocatechuic acid, quercetin, quercitrin, rutin, swertiamarin, terpineol, trans-cinnamic acid, umbelliferone, and umbellic acid.

16. The dermal cytochrome P450 1A (CYP1A) enhancer according to claim 14, wherein said enhancer is at least one selected from the group consisting of(−)-epicatechin, cineole, narigin, and protocatechuic acid.
